(19) United States
(12) Patent Application Publication
(10) Pub. No.: US 2006/0045846 A1
(43) Pub. Date: Mar. 2, 2006

(54) Reagents and methods for labeling terminal olefins

(76) Inventors: Thomas E. Horstmann, Boston, MA (US); Bryan M. Lewis, Andover, MA (US)

Correspondence Address: CHOATE, HALL & STEWART LLP, TWO INTERNATIONAL PLACE, BOSTON, MA, 02110, US

(21) Appl. No.: 10/929,624

(22) Filed: Aug. 30, 2004

(51) Int. Cl. A61K 51/00

(52) U.S. Cl. 424/1.65; 534/11

(57) ABSTRACT

In one aspect, the present invention provides a method for labeling a terminal olefin, the method comprising a step of treating a terminal olefin substrate having the structure: 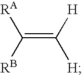
with a labeled ethylene reagent in the presence of a suitable catalyst under suitable olefin metathesis reaction conditions to form a labeled terminal olefin having the structure:

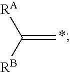

wherein R^{A} and R^{B} are independently hydrogen, or an aliphatic, alicyclic, heteroaliphatic, heterocyclic, aryl or heteroaryl moiety, with the proviso that R^{A} and R^{B} are not each hydrogen, or R^{A} and R^{B} taken together with the carbon atom to which they are attached form an alicyclic or heterocyclic moiety; and * denotes the presence of an isotopic label on the terminal carbon atom.

### BACKGROUND OF THE INVENTION

Stable isotopes (e.g., deuterium, ¹³C, ¹⁵N, ¹⁸O) are nonradioactive isotopes which contain one additional neutron than the normally abundant isotope of the atom in question. Deuterated compounds have been used in pharmaceutical research to investigate the in vivo metabolic fate of the compounds by evaluation of the mechanism of action and metabolic pathway of the non deuterated parent compound. (Blake et al. J. Pharm. Sci. 64, 3, 367-391, 1975). Such metabolic studies are important in the design of safe, effective therapeutic drugs, either because the in vivo active compound administered to the patient or because the metabolites produced from the parent compound prove to be toxic or carcinogenic (Foster et al., Advances in drug Research Vol. 14, pp. 2-36, Academic press, London, 1985)

Radioisotopes find use in a variety of biomedical applications. For example, radioisotopes may be used for biochemical analyses (e.g., biochemical analysis, diagnostics, radiotherapy). The presence of some radioactive materials may be readily detected even when they exist in very low concentrations. Radioisotopes can therefore be used to label molecules of biological samples in vitro. Pathologists have devised hundreds of tests to determine the constituents of blood, serum, urine, hormones, antigens and many drugs by means of associated radioisotopes. These procedures are known as radioimmuno assays and, although the biochemistry is complex, kits manufactured for laboratory use are very easy to use and give accurate results. In addition, diagnostic techniques in nuclear medicine use radioactive tracers which emit gamma rays from within the body. These tracers are generally short-lived isotopes linked to chemical compounds which permit specific physiological processes to be scrutinised. They typically come in the form of radionuclide which can be given by injection, inhalation or orally. Radioisotopes may also find use in radiotherapy. These typically involve radioisotopes such as ¹³¹I, ¹⁹²Ir, ⁸⁹Sr, ¹⁵³Sm and ¹⁸⁶Re.

Methods for labeling compounds (e.g., with stable or radioactive isotopes) may be classified into four main categories. Specific labeling yields molecules where the isotopes occupy known specific positions without any ambiguity. Uniform labeling yields the labeled molecules in which the isotopes are distributed in a statistically uniform pattern. General labeling yields the molecules where the isotopes are distributed in a general or random pattern, not always known with any certainty. Nominal labeling is used to indicate the position of the isotopes where there is uncertainty as to whether the labeling is confined to the positions specified.

Current practical methods for isotopically labeling compounds fall into three main categories: (a) Chemical syntheses, (b) Biochemical methods and (c) Isotope exchange reactions.

Biochemical methods employ either a purified or partially purified enzyme, or intact organism or cells. These methods are effective and important for labeling a range of C-14 labeled compounds widely used in tracer applications including L-amino acids, carbohydrates, nucleosides and nucleotides. These compounds are readily available in their natural configurations, uniformly labeled, by growing algae on [¹⁴C]carbon dioxide or by photosynthesis in detached plant leaves. On the other hand, biosynthetic labeling with tritium has proved of limited practical use, due mainly to the limitations imposed by radiation effects as well as isotope exchange.

In isotope exchange reactions an atom in a molecule is substituted by its radioactive equivalent. The reactions are reversible. They are extremely important and are widely used for isotopic labeling with tritium, although they have very limited practical application to isotopic labeling with C-14. An attractive method for isotopically labeling compounds remains chemical synthesis, which allows greater flexibility in terms of controlling the specificity of labeling. Syntheses of C-14 labeled compounds tend to follow the broad lines of classical organic chemistry albeit on a small chemical scale. For example, barium [¹⁴C]carbonate or [¹⁴C]carbon dioxide are commonly used as starting materials from which the labeled atom(s) is usually derived. A number of useful intermediates are prepared by reduction reactions. Radio chemical syntheses with tritium are generally one or two stage reactions and are usually much less complex than those used for isotopic labeling with C-14. In addition, tritium is a relatively low cost isotope by comparison with C-14 and radiochemical yields are therefore less important for tritium labeled compounds than for C-14 labeled compounds. Starting materials are tritium gas, tritiated water or tritiated metal hydrides.

As discussed above, a significant advantage of chemical synthesis of a labeled compound is the ability to control the specificity of labeling. This is usually unambiguous in the case of C-14 labeled compounds from the synthetic route chosen. However, it is important to remember that non-specific hydrogen-tritium exchange is always a possibility in the presence of metal hydrogen transfer catalysts such as Pt or Pd. An example to illustrate this point is the preparation of tritiated folic acid by catalyzed halogen-tritium replacement from 3′,5′-dibromofolic acid. The non-specific isotopic substitution could present a serious problem in some applications of tritium labeled compounds as tracers. Therefore, confirmation of the tritium-labeling site by tritium NMR is required. See Evans et al., J. Labelled Compd. Radiopharm., 1979, 16, 697.

There remains a need to develop efficient and high yielding synthetic methodologies to isotopically label compounds (e.g., pharmaceuticals) with stable, as well as radioactive isotopes. In particular, synthetic methods for specific labeling of compounds are needed.

### SUMMARY OF THE INVENTION

In one aspect, the present invention provides a method for labeling a terminal olefin, the method comprising a step of treating a terminal olefin substrate having the structure:

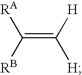

with a labeled ethylene reagent in the presence of a suitable catalyst under suitable olefin metathesis reaction conditions to form a labeled terminal olefin having the structure:

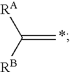

wherein R^{A} and R^{B} are independently hydrogen, or an aliphatic, alicyclic, heteroaliphatic, heterocyclic, aryl or heteroaryl moiety, with the proviso that R^{A} and R^{B} are not each hydrogen, or R^{A} and R^{B} taken together with the carbon atom to which they are attached form an alicyclic or heterocyclic moiety; and

* denotes the presence of an isotopic label on the terminal carbon atom.

In certain embodiments, the ethylene reagent is ethylene-d4 and the labeled terminal olefin has the structure:

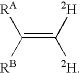

In certain embodiments, the ethylene reagent is ethylene-³H₄ and the labeled terminal olefin has the structure:

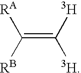

In certain embodiments, the ethylene reagent is ethylene-1,2-¹³C₂ and the labeled terminal olefin has the structure:

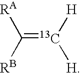

In certain embodiments, the ethylene reagent is ethylene-1,2-¹⁴C₂ and the labeled terminal olefin has the structure:

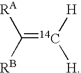

In certain embodiments, 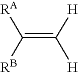
represents a pharmaceutically/therapeutically useful compound. Such compound may be an FDA approved drug, a prodrug, a clinical trial candidate, a lead compound, or a compound at early stages of Research & Development drug discovery program. In certain exemplary embodiments, 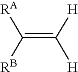
represents one of the compounds depicted in Appendix A, either in free form, known salt form thereof, or any stable salt form the particular compound may be made to exist. In certain exemplary embodiments, 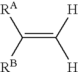
is alminoprofen, amisometradine, dicryl, ethacrynic acid, ethalfluralin, methallatal, rhodinol, acetamidoeugenol, albutoin, alclofenac, alibendol, allethrin I, allethrin II, allocupreide sodium, allylestrenol, almitrine, aloxidone, alpiropride, alprenolol, altmogest, arninometradine, apiole, aprobarbital, apronalide, bialamicol, butalbital, buthalital sodium, cabergoline, enallylpropymal, enilconazole, eugenol, gravitol, honokiol, isophytol, levallorphan, nalorphine, naloxone, nealbarbital, penicillin O., phenallymal, proxibarbal, rocuronium, safrole, secobarbital sodium, tacrolimus, talbutal, talipexole, thiamylal, valdetamide, veralipride, vigabatrin, verteporfin, bexarotene, calcipotriol, cefdinir, cefixime, exemestane, nalmefene, doxercalciferol, or a compound having the structure:

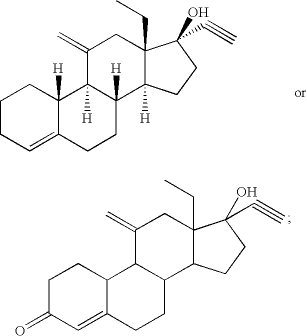

or salt thereof.

In certain embodiments, 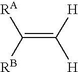
represents a halichondrin-type compound having the structure:

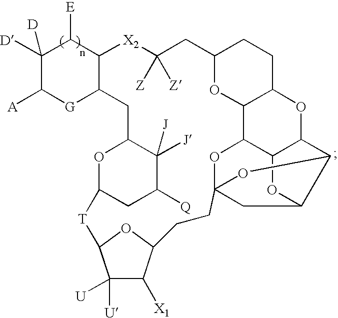

wherein A, D, D′, E, G, J, J′, T, U, U′, Q, X₁, X₂, Z, Z′ and n are as defined in classes and subclasses herein. In certain embodiments, the halichondrin-type compound has the structure:

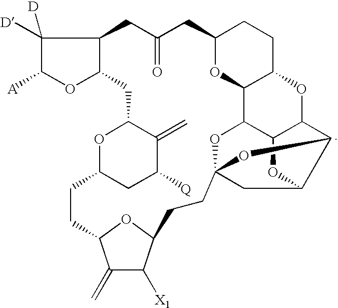

### DEFINITIONS

In accordance with the present invention and as used herein, the following terms, are defined with the following meanings, unless explicitly stated otherwise.

Certain compounds disclosed in the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether)), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having about 1-6 carbon atoms.

In certain embodiments, the alkyl, alkenyl and alkynyl groups employed in the invention contain about 1-20 aliphatic carbon atoms. In certain other embodiments, the alkyl, alkenyl, and alkynyl groups employed in the invention contain about 1-10 aliphatic carbon atoms. In yet other embodiments, the alkyl, alkenyl, and alkynyl groups employed in the invention contain about 1-8 aliphatic carbon atoms. In still other embodiments, the alkyl, alkenyl, and alkynyl groups employed in the invention contain about 1-6 aliphatic carbon atoms. In yet other embodiments, the alkyl, alkenyl, and alkynyl groups employed in the invention contain about 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl-n, cyclohexyl, —CH₂-cyclohexyl, cyclohexenylethyl, cyclohexanylethyl, norborbyl moieties and the like, which again, may bear one or more substituents.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, heteroaliphatic or heterocyclic moieties, may optionally be substituted. An analogous convention applies to other generic terms such as “cycloalkenyl”, “cycloalkynyl” and the like.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be branched or linear unbranched. In certain embodiments, heteroaliphatic moieties are substituted by independent replacement of one or more of the hydrogen atoms thereon with one or more moieties including, but not limited to aliphatic; heteroaliphatic; alicyclic; heteroalicyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃;—or—GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heteroalicyclic, aromatic, heteroaromatic, aryl, heteroaryl, alkylaryl, or alkylheteroaryl moiety. Additional examples of generally applicable substituents are illustrated by the specific embodiments shown in the Examples that are described herein.

The term “heteroalicyclic”, “heterocycloalkyl” or “heterocyclic”, as used herein, refers to compounds which combine the properties of heteroaliphatic and cyclic compounds and include but are not limited to saturated and unsaturated mono- or polycyclic heterocycles such as morpholino, pyrrolidinyl, furanyl, thiofuranyl, pyrrolyl etc., which are optionally substituted with one or more functional groups, as defined herein. In certain embodiments, the term “heterocyclic” refers to a non-aromatic 5-, 6- or 7- membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; alicyclic; heteroalicyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; —or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(⊚O)NR^{G2}—, —C(═S)—, —C(═S)S—, —SC(═S)—, —SC(═S)S—, —C(⊚NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heteroalicyclic, aromatic, heteroaromatic, aryl, heteroaryl, alkylaryl, or alkylheteroaryl moiety. Additional examples or generally applicable substituents are illustrated by the specific embodiments shown in the Examples, which are described herein.

Additionally, it will be appreciated that any of the alicyclic or heteroalicyclic moieties described above and herein may comprise an aryl or heteroaryl moiety fused thereto. Additional examples of generally applicable substituents are illustrated by the specific embodiments shown in the Examples that are described herein.

In general, the term “aromatic moiety”, as used herein, refers to stable substituted or unsubstituted unsaturated mono- or polycyclic hydrocarbon moieties having preferably 3-14 carbon atoms, comprising at least one ring satisfying the Huckel rule for aromaticity. Examples of aromatic moieties include, but are not limited to, phenyl, indanyl, indenyl, naphthyl, phenanthryl and anthracyl.

In general, the term “heteroaromatic moiety”, as used herein, refers to stable substituted or unsubstituted unsaturated mono-heterocyclic or polyheterocyclic moieties having preferably 3-14 carbon atoms, comprising at least one ring satisfying the Huckel rule for aromaticity. Examples of heteroaromatic moieties include, but are not limited to, pyridyl, quinolinyl, dihydroquinolinyl, isoquinolinyl, quinazolinyl, dihydroquinazolyl, and tetrahydroquinazolyl.

It will also be appreciated that aromatic and heteroaromatic moieties, as defined herein, may be attached via an aliphatic (e.g., alkyl) or heteroaliphatic (e.g., heteroalkyl) moiety and thus also include moieties such as -(aliphatic)aromatic, -(heteroaliphatic)aromatic, -(aliphatic)heteroaromatic, -(heteroaliphatic)heteroaromatic, -(alkyl)aromatic, (heteroalkyl)aromatic, -(alkyl)heteroaromatic, and -(heteroalkyl)heteroaromatic moieties. Thus, as used herein, the phrases “aromatic or heteroaromatic moieties” and “aromatic, heteroaromatic, -alkyl)aromatic, -(heteroalkyl)aromatic, -(heteroalkyl)heteroaromatic, and (heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

In general, the term “aryl” refers to aromatic moieties, as described above, excluding those attached via an aliphatic (e.g., alkyl) or heteroaliphatic (e.g., heteroalkyl) moiety. In certain embodiments of the present invention, “aryl” refers to a mono- or bicyclic carbocyclic ring system having one or two rings satisfying the Huckel rule for aromaticity, including, but not limited to, phenyl, naphthyl, tetrahydronaphthyl, indanyl, indenyl and the like.

Similarly, the term “heteroaryl” refers to heteroaromatic moieties, as described above, excluding those attached via an aliphatic (e.g., alkyl) or heteroaliphatic (e.g., heteroalkyl) moiety. In certain embodiments of the present invention, the term “heteroaryl”, as used herein, refers to a cyclic unsaturated radical having from about five to about ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

Substituents for aryl and heteroaryl moieties include, but are not limited to, any of the previously mentioned substitutents, i.e., the substituents recited for aliphatic moieties, or for other moieties as disclosed herein, resulting in the formation of a stable compound. For example, aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; alicyclic; heteroalicyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃;—or -GR^{G1} wherein G is —O—, —S—, —NR^{G2}—, —C(═O)—, —(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heteroalicyclic, aromatic, heteroaromatic, aryl, heteroaryl, alkylaryl, or alkylheteroaryl moiety. Additional examples of generally applicable substituents are illustrated by the specific embodiments shown in the Examples that are described herein.

The terms “alkoxy” (or “alkyloxy”), as used herein refers to an alkyl group, as previously defined, attached to the parent molecular moiety through an oxygen atom (“alkoxy”). In certain embodiments, the alkyl group contains about 1-20 aliphatic carbon atoms. In certain other embodiments, the alkyl group contains about 1-10 aliphatic carbon atoms. In yet other embodiments, the alkyl group contains about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkoxy groups, include but are not limited to, methoxy, ethoxy, propoxy, isopropoxy, n-butoxy, tert-butoxy, neopentoxy and n-hexoxy, and the like.

The term “amine” refers to a group having the structure —N(R)₂ wherein each occurrence of R is independently hydrogen, or an aliphatic, heteroaliphatic, aromatic or heteroaromatic moiety, or the R groups, taken together, may form a heterocyclic moiety.

The term “alkylamino” refers to a group having the structure —NHR′ wherein R′ is alkyl, as defined herein. The term “aminoalkyl” refers to a group having the structure NH₂R′—, wherein R′ is alkyl, as defined herein. In certain embodiments, the alkyl group contains about 1-20 aliphatic carbon atoms. In certain other embodiments, the alkyl group contains about 1-10 aliphatic carbon atoms. In yet other embodiments, the alkyl, alkenyl, and alkynyl groups employed in the invention contain about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkylamino include, but are not limited to, methylamino, ethylamino, iso-propylamino and the like.

The terms “halo” and “halogen” as used herein refer to an atom selected from fluorine, chlorine, bromine and iodine. In certain embodiments, the term “halogen” encompasses fluorine, chlorine, bromine and iodine and their isotopes.

The term “haloalkyl” denotes an alkyl group, as defined above, having one, two, or three halogen atoms attached thereto and is exemplified by such groups as chloromethyl, bromoethyl, trifluoromethyl, and the like.

The term “acyl”, as used herein, does not substantially differ from the common meaning of this term in the art, and refers to a moiety of structure —C(O)Rₓ, wherein Rₓ is a substituted or unsubstituted, aliphatic, alicyclic, heteroaliphatic, heteroalicyclic, aryl or heteroaryl moiety.

The term “imine”, as used herein, does not substantially differ from the common meaning of this term in the art, and refers to a moiety of structure —C(═NRₓ)R_{y}, wherein Rₓ is hydrogen or an optionally substituted aliphatic, alicyclic, heteroaliphatic, heteroalicyclic, aryl or heteroaryl moiety; and R_{y} is an optionally substituted aliphatic, alicyclic, heteroaliphatic, heteroalicyclic, aryl or heteroaryl moiety.

As used herein, * in the chemical structure: 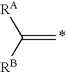
denotes the presence of an isotopic label on the terminal carbon atom. Therefore, the designation 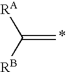
throughout this document is not limited to: 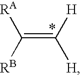
where * designates, for example, ¹³C or ¹⁴C. Rather, the structure encompasses olefins where the terminal carbon atom (i.e., right-hand side carbon atom) either is an isotope of carbon (e.g., ¹¹C, ¹³C, ¹⁴C), or bears at least one isotopic atom (e.g., deuterium, tritium, ¹⁴O, ¹⁸F, ⁷⁶Br, ¹²³I, ¹²⁵I, ¹³¹I) or both.

Similarly, as used herein, the chemical structure: 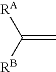
is not to be interpreted as solely designating the entity having the structure: 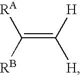
as commonly understood in the chemical art. Rather, 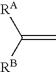
signifies a terminal olefin where the terminal carbon atom is unlabeled (i.e., one that does not bear an isotopic label). In this context, “terminal carbon atom” refers to the ethylenic carbon atom in the structure 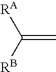
above where substituents are unspecified. In certain embodiments, the subtituents on the terminal carbon atom are H, F, Cl, Br or I.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heteroalicyclic”, “heterocycloalkyl”, “heterocycle”and the like encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aryl”, “heteroaryl”and the like encompass both substituted and unsubstituted groups.

Some examples of substituents of the above-described aliphatic (and other) moieties of compounds of the invention include, but are not limited to aliphatic; heteroaliphatic; alicyclic; heteroalicyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; —or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(=NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heteroalicyclic, aromatic, heteroaromatic, aryl, heteroaryl, alkylaryl, or alkylheteroaryl moiety. Additional examples of generally applicable substituents are illustrated by the specific embodiments shown in the Examples that are described herein.

### DETAILED DESCRIPTION OF CERTAIN PREFERRED EMBODIMENTS OF THE INVENTION

As discused above, there remains a need to develop efficient and high yielding synthetic methods for specific labeling of compounds (e.g., pharmaceuticals) with stable, as well as radioactive isotopes. In one aspect, the present invention provides a method for specific labeling of terminal olefins via olefin metathesis.

Olefin metathesis is a carbon-carbon bond breaking/bond making process which involves overall exchange of double bond moieties between two olefins. Traditionally, olefin metathesis reactions may be classified in three main categories, as illustrated in Scheme 1.

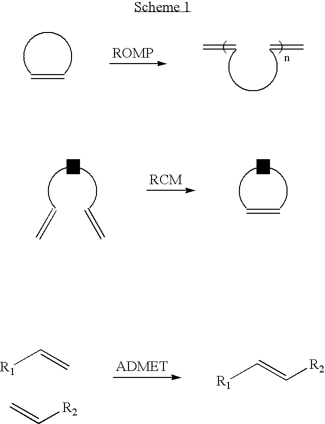

Ring-opening metathesis polymerization (ROMP) involves the formation of polyolefins from strained cyclic olefins; ring-closing metathesis (RCM) involves the intramolecular transformation of an alpha, omega-diene to a cyclic olefin; and acyclic diene metathesis (ADMET) involves the intermolecular exchange of olefins.

Olefin metathesis has significant potential not only in the area of preparative, organic synthesis (RCM, ethenolysis, metathesis of acyclic olefins) but also in polymer chemistry (ROMP, ADMET, alkyne polymerization). Its discovery in the 1950s lead to the development of several industrial processes (Reviews: Ivin, K. J.; Mol, J. C. Olefin Metathesis and Metathesis Polymerization, Academic Press, New York, 1997; Schuster, M. et al., Angew. Chem. 1997, 109, 2125). Nevertheless, olefin metathesis did not develop into a broadly applicable synthetic method until the recent discovery of new catalysts (J.C. Mol in: B. Cornils, W. A. Herrmann: Applied Homogeneous Catalysis with Organometallic Compounds, VCH, Weinheim, 1996, p. 318-332; M. Schuster, S. Blechert, Angew. Chem. 1997, 109, 2124-2144; Angew. Chem. Int. Ed. Engl. 1997, 36, 2036-2056).

In one aspect, the present invention provides the first instance of application of olefin metathesis methodology to isotopic labeling (e.g., radiolabeling) of terminal olefins. In one aspect, the present invention provides a method for labeling a terminal olefin, the method comprising a step of treating a terminal olefin substrate having the structure:

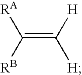

with a labeled ethylene reagent in the presence of a suitable catalyst under suitable olefin metathesis reaction conditions to form a labeled terminal olefin having the structure:

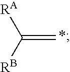

wherein R^{A} and R^{B} are independently hydrogen, or an aliphatic, alicyclic, heteroaliphatic, heterocyclic, aromatic or heteroaromatic moiety, with the proviso that R^{A} and R^{B} are not each hydrogen, or R^{A} and R^{B} taken together with the carbon atom to which they are attached form an alicyclic or heterocyclic moiety; and

* denotes the presence of an isotopic label on the terminal carbon atom.

In certain embodiments, R^{A} and R^{B} are independently hydrogen, or an aliphatic, alicyclic, heteroaliphatic, heterocyclic, aryl or heteroaryl moiety, with the proviso that R^{A} and R^{B} are not each hydrogen, or R^{A} and R^{B} taken together with the carbon atom to which they are attached form an alicyclic or heterocyclic moiety.

In certain embodiments, more than one metathesis cycle may be desired to obtain the target conversion rate. In certain embodiments, the terminal olefin substrate is subject to two metathesis cycles. In certain embodiments, the terminal olefin substrate is subject to three metathesis cycles. In certain embodiments, the terminal olefin substrate is subject to four metathesis cycles.

In another aspect, the present invention provides a method for labeling a terminal olefin, the method comprising steps of:

(a) treating a terminal olefin substrate having the structure:

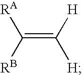

with a labeled ethylene reagent in the presence of a suitable catalyst under suitable olefin metathesis reaction conditions to yield a reaction mixture comprising a labeled terminal olefin having the structure:

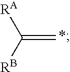

and unreacted terminal olefin substrate;

wherein R^{A} and R^{B} are independently hydrogen, or an aliphatic, alicyclic, heteroaliphatic, heterocyclic, aromatic or heteroaromatic moiety, with the proviso that R^{A} and R^{B} are not each hydrogen, or R^{A} and R^{B} taken together with the carbon atom to which they are attached form an alicyclic or heterocyclic moiety;

* denotes the presence of an isotopic label on the terminal carbon atom; and

(b) repeating step (a) using the reaction mixture as a substrate, thereby reducing the amount of unreacted terminal olefin substrate;

(c) optionally repeating step (b) until the ratio [labeled terminal olefin]/[unreacted terminal olefin substrate] reaches a desired value.

In certain embodiments, the amount of unreacted unlabeled material is a function of reaction cycles through which the terminal olefin substrate is put. Presumably, after enough cycles the amount of unlabeled (i.e., unreacted) terminal olefin substrate could be reduced to zero. However, this is probably dependent on the identity and/or concentration of the terminal olefin substrate, labeled ethylene reagent and/or catalyst.

In certain embodiments, neither R^{A} nor R^{B} comprises an olefin moiety. In certain other embodiments, neither R^{A} nor R^{B} comprises a disubstituted olefin moiety.

In certain embodiments, R^{A} and R^{B} are independently hydrogen, or an aliphatic, alicyclic, heteroaliphatic, heterocyclic, aryl or heteroaryl moiety, with the proviso that R^{A} and R^{B} are not each hydrogen, or R^{A} and R^{B} taken together with the carbon atom to which they are attached form an alicyclic or heterocyclic moiety.

In certain embodiments, metathesis reaction (e.g., cleavage and/or isotopic labeling) at R^{A} and/or R^{B} is desired, and thus R^{A} and/or R^{B} comprises an alkenyl or cycloalkenyl moiety. In certain embodiments, the alkenyl or cycloalkenyl moiety in R^{A} and/or R^{B} is cleaved in the course of the olefin metathesis reaction ultimately converting the internal olefinic carbon of R^{A} and/or R^{B} to a terminal labeled carbon atom. In certain embodiments, an ethylene reagent symmetrically labeled at both carbon atoms (i.e., each carbon atom bears the same label) is used, and cleavage of the olefin moiety at R^{A} and/or R^{B} proceeds with concomittant labeling of the substrate at the site of the R^{A}/R^{B} olefin. In certain other embodiments, an ethylene reagent labeled at only one of its carbon atoms is used, and cleavage of the olefin moiety at R^{A} and/or R^{B} results in a mixture of labeled and unlabeled substrate at the site of the R^{A}/R^{B} olefin. For example, the method may be used with terminal olefin-containing substrates bound to a solid support via a suitably selected olefin containing linker (e.g., disubstituted olefin, or olefin with substitution pattern favorable to metathesis cleavage). In certain embodiments, the method would achieve (i) labeling of the substrate's terminal olefin, (ii) release of the substrate from the solid support, and (iii) depending on the labeled ethylene reagent (e.g., reagent symmetrically labeled at both carbon atoms, or labeled at only one of its carbon atoms), labeling at the cleavage site.

In certain embodiments, the ethylene reagent is symmetrically labeled at both carbon atoms (i.e., each carbon atom bears the same label), and the method yields the desired labeled terminal olefin 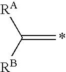
as primary product. One of ordinary skill in the art will recognize that the metathesis reaction may not proceed with a 100% yield. Therefore, some unreacted (unlabeled) terminal olefin 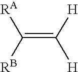
may be present.

One of ordinary skill in the art will also appreciate that, the labeled terminal olefin 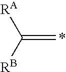
obtained by practicing the inventive method may exist as a mixture of stereoisomers. For example, if the symmetrically labeled ethylene reagent has the structure: 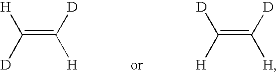
the method will yield 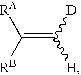
where the olefin geometry is undetermined (e.g., mixture of geometric isomers).

In certain other embodiments, the ethylene reagent is labeled at only one of its carbon atoms, and the method yields a mixture of labeled terminal olefin 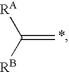
and unlabeled terminal olefin 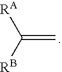
It is to be understood that 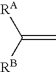
does not necessarily designate the entity having the structure: 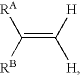
as commonly understood in the chemical art. Rather, 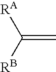
signifies that the terminal olefinic carbon atom inherits the subtitution pattern originally present on the unlabeled carbon atom of the ethylene reagent. For example, if 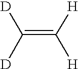
is used as labeled ethylene reagent, 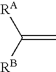
designates 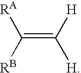
If 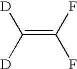
is used as labeled ethylene reagent, 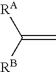
designates

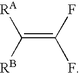

The present invention encompasses methods where R^{A} and R^{B} are any moiety that is tolerated by the olefin metathesis reaction conditions. For example, R^{A} and R^{B} are preferably substantially chemically inert with respect to olefin metathesis reaction conditions (e.g., chemical functionalities present on R^{A} and R^{B} do not substantially affect, negatively impact or otherwise interfere with the olefin metathesis reaction). Examples of suitable functionalities that may be present on R^{A} and R^{B} include, but are not limited to, electron withdrawing groups, electron donating groups, sterically hindered groups, aromatic groups. Other suitable groups will be readily apparent to the skilled practitioner from metathesis reaction conditions known in the art. In certain embodiments, R^{A} and R^{B} do not comprise an alkenyl or cycloalkenyl moiety.

In certain embodiments, neither R^{A} nor R^{B} comprises an olefin moiety. In certain other embodiments, neither R^{A} nor R^{B} comprises a disubstituted olefin moiety. In certain embodiments, R^{A} and/or R^{B} comprises a tri- or tetra-substituted olefin moiety.

In certain embodiments, R^{A} and R^{B} are independently alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, aryl, heteroaryl, -(alkyl)aryl, -(alkenyl)aryl, -(alkynyl)aryl, -(heteroalkyl)aryl, -(heteroalkenyl)aryl, -(heteroalkynyl)aryl, -(alkyl)heteroaryl, -(alkenyl)heteroaryl, -(alkynyl)heteroaryl, -(heteroalkyl)heteroaryl, -(heteroalkenyl)heteroaryl, -(heteroalkynyl)heteroaryl, or R^{A} and R^{B} taken together with the carbon atom to which they are attached form an alicyclic or heterocyclic moiety. In certain embodiments, any olefin moiety present in the alkenyl, cycloalkenyl, heteroalkenyl, heterocycloalkenyl, -(alkenyl)aryl, -(alkenyl)aryl, -(heteroalkenyl)aryl, -(alkenyl)heteroaryl, -(heteroalkenyl)heteroaryl referred to directly above is a tri- or tetra-substituted olefin moiety.

In certain embodiments, R^{A} and R^{B} are independently alkyl, alkynyl, cycloalkyl, cycloalkynyl, heteroalkyl, heteroalkynyl, heterocycloalkyl, heterocycloalkynyl, aryl, heteroaryl, -(alkyl)aryl, -(alkynyl)aryl, -(heteroalkyl)aryl, -(heteroalkynyl)aryl, -(alkyl)heteroaryl, -(alkynyl)heteroaryl, -(heteroalkyl)heteroaryl, -(heteroalkynyl)heteroaryl, or R^{A} and R^{B} taken together with the carbon atom to which they are attached form an alicyclic or heterocyclic moiety. In certain embodiments, the alicyclic or heterocyclic moiety is saturated (i.e., does not comprise an olefin).

In certain embodiments, the catalyst is any reagent known in the art to effect olefin metathesis. Olefin metathesis reactions are catalyzed by various metal compounds. Many of the classical catalysts consist of mixtures of various components, they are ill defined in their chemical composition, show a poor compatibility with functional groups and are inefficient as a consequence of little active species present. More modern catalysts or catalyst precursors with a better application profile comprise complexes of the general types I-IX, as depicted in Scheme 2 (references: type I (M=Ru, Os): WO 96/04289, 15.02.1996; Nguyen S. T. et al. J. Am. Chem. Soc. 1992, 114, 3974; Nguyen S. T. et al. J. Am. Chem. Soc. 1993, 115, 9858; Schwab, P. et al. Angew. Chem. 1995, 107, 2179 (Angew. Chem. Int. Ed. Engl., 1995, 34, 2039); Schwab, P. et al. J. Am. Chem. Soc. 1996, 118, 100; Mohr, B. et al. Organometallics 1996, 15, 4317; Wilhelm, T. E. et al. Organometallics 1997, 16, 3867; Belderrain, T. R. Organometallics 1997, 16, 4001. Type II (M=Mo, W): Schrock, R. R. et al. J. Am. Chem. Soc. 1990, 112, 3875; Fujimura, O. et al. Organometallics 1996, 15, 1865. Type III: Quignard, F. et al. J. Mol. Catal. 1986, 36, 13. Type IV (M=Nb, Ta): Rocklage, S. M. et al. J. Am. Chem. Soc. 1981, 103, 1440; Wallace, K. C. et al. Macromolecules 1987, 20, 448. Type V (cp=cyclopentadienyl or substituted cyclopentadienyl): U.S. Pat. No. 4,567,244, Jan. 28, 1986. Type VI: Herrmann, W. A. et al. Angew. Chem. 1991, 103, 1704. Type VII: Nugent, W. A. et al. J. Am. Chem. Soc. 1995, 117, 8992. Type VIII: Davie E. S. J. Catal. 1972, 24, 272. Type IX: Herrmann, W. A. et al. Angew. Chem. 1996, 108, 1169.)

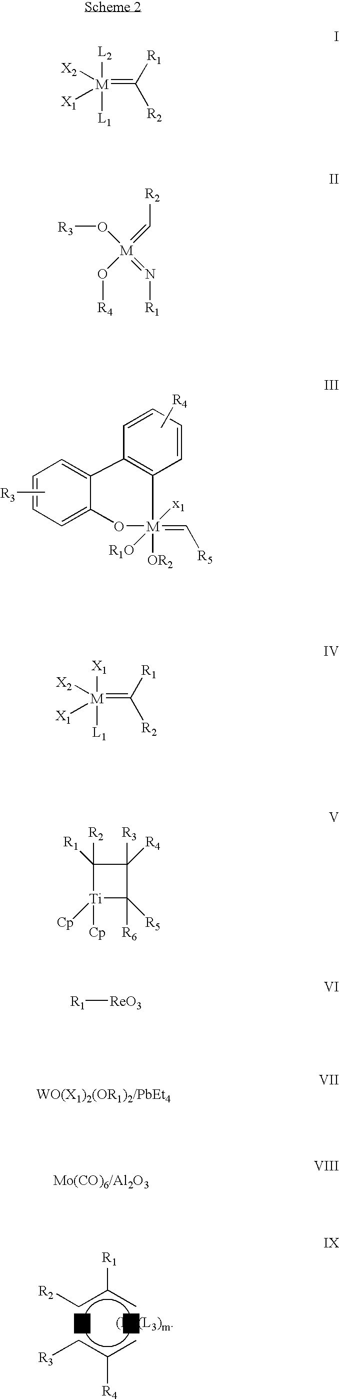

Additional guidance can be found inter alia in U.S. Pat. Nos. 6,696,597; 6,635,768; 6,624,265; 6,605,748; 6,590,048; 5,312,940; 5,342,909; 5,728,917; 5,750,815; 5,710,298 and 5,831,108 and PCT Publications WO 97/20865 and WO 97/29135 which are all incorporated herein by reference. For representative metathesis catalysts references, see: (a) Feldman, J.; Schrock, R. R. In Progress in Inorganic Chemistry; Lippard, S. J., Ed.; John Wiley & Sons, Inc.: New York, 1991; Vol. 39, pp 1-74. (b) Grubbs, R. H.; Pine, S. H. In ComprehensiVe Organic Synthesis; Trost, B. M., Fleming, I., Eds.; Pergamon: New York, 1991; Vol. 5, Chapter 9.3. (c) Schwab, P.; Grubbs, R. H.; Ziller, J. W. J. Am. Chem. Soc. 1996, 118, 100-110. (d) Weck, M.; Schwab, P.; Grubbs, R. H. Macromolecules 1996, 29, 1789-1793. (e) Nugent, W. A.; Feldman, J.; Calabrese, J. C. J. Am. Chem. Soc. 1995, 117, 8992-8998. (f) Nguyen, S. T. Ph.D. Thesis, California Institute of Technology, January 1995. (g) Wu, Z.; Nguyen, S. T.; rubbs, R. H.; Ziller, J. W. J. Am. Chem. Soc. 1995, 117, 5503-5511. (h) Wache, S.; Herrmann, W. A.; Artus, G.; Nuyken, O.; Wolf, D. J. Organomet. Chem. 1995, 491, 181-188. (i) Grubbs, R. H. J. Macromol. Sci., Pure Appl. Chem. 1994, A31, 1829-1833. (1) Schrock, R. R. Pure Appl. Chem. 1994, 66, 1447-1454. (k) Nguyen, S. T.; Grubbs, R. H. J. Am. Chem. Soc. 1993, 115, 9858-9859. (1) Couturier, J.-L.; Tanaka, K.; Leconte, M.; Basset, J.-M.; Ollivier, J. Angew. Chem., Int. Ed. Engl. 1993, 32, 112-114. The skilled practitioner will know how to selected a catalyst known in the art suitable for effecting the reaction.

In certain embodiments, the invention may be practiced using one or more of the following commercially available catalysts:

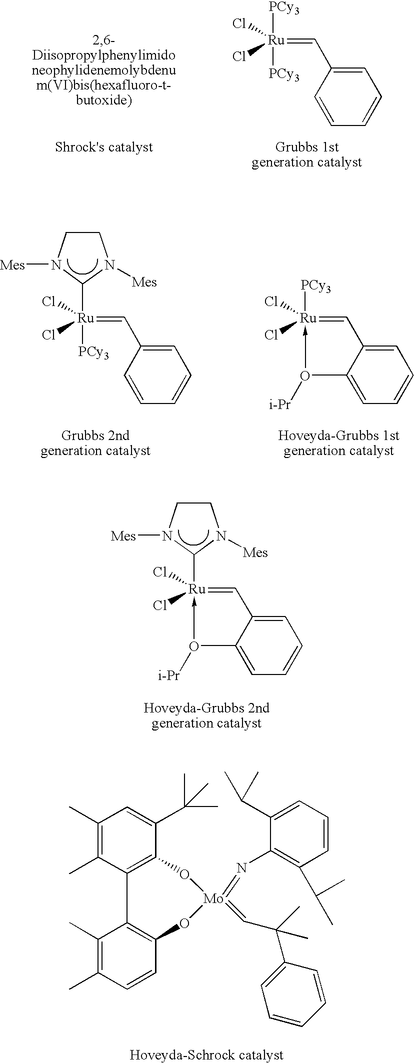

In certain embodiments, the ethylene reagent is ethylene-d4 and the labeled terminal olefin has the structure:

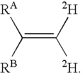

In certain embodiments, the ethylene reagent is 1,2-ethylene-d2 and the labeled terminal olefin has the structure:

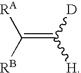

In certain embodiments, the ethylene reagent is ethylene-3H4 and the labeled terminal olefin has the structure:

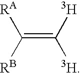

In certain embodiments, the ethylene reagent is an isotopically labeled fluoroethylene derivative. 1,1-Difluoroethylene-¹⁹F₂ have been reported in Angewandte Chemie Int. Ed. Engl., 2001, 40(18), 3441. Radiolabeled equivalents may be obtained by substituting ¹⁹F for ¹⁸F. For example, in certain embodiments, the ethylene reagent is 1,1-difluoroethene-¹⁸F₂ and the method yieds a mixture of

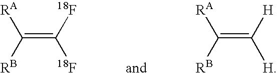

In certain embodiments, the ethylene reagent is 1,2-difluoroethylene-¹⁸F₂ and the labeled terminal olefin has the structure:

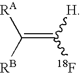

The following provide exemplary synthetic guidance for the preparation of ¹⁸F-containing ethylene derivatives:

(i) Tetrafluoroethylene: a single fluorine-19 replaced with F-18 [cas # 80281-24-9]. Van der Linde, K. D.; Spoelstra-Van Balen, S.; Kaspersen, F. M. The chemistry of fluorine-18-recoil atoms in gaseous tetrafluoroethane scavenged with hydrogen sulfide. Radiochemical and Radioanalytical Letters (1981), 49(4), 239-50; (ii) Tetrafluoroethylene: a single fluorine-19 replaced with F-18 [cas # 80281-24-9].

(ii) 1,1-difluoroethylene: one of the fluorine-19 is replaced with F-18 [cas# 64429-61-4] or 1,1,2-trifluoroethylene: the 2-F19 is replaced with 2-F18 [64429-60-3]. Manning, Ronald G.; Root, John W. Chemistry of nuclear recoil fluorine-18 atoms. 10. Studies of fluorine-18 caged capture processes in 1,1,1-trifluoroethane/hydrogen sulfide and 1,1-difluoroethane/hydrogen sulfide liquid mixtures. Journal of Physical Chemistry (1977), 81(25), 2576-86.

(iii) Fluoroethylene: the F-19 is replaced with F-18 [3791-37-5]. Williams, Ronald L.; Rowland, F. S. Addition of fluorine-18 atoms to acetylene. Journal of the American Chemical Society (1972), 94(4), 1047-51.

(iv) Williams, Ronald L.; Rowland, F. S. Reactions of fluorine-18 atoms with ethylene. Journal of Physical Chemistry (1972), 76(24), 3509-17.

(v) Rogers, Patricia J.; Selco, Jodye I.; Rowland, F. S. Non-RRKM decomposition of chemically activated radicals: reaction of fluorine atoms with tetraallylgermanium. Chemical Physics Letters (1983), 97(3), 313-16.

(vi) Di Raddo, Pasquale; Diksic, Mirko. Mild and regiospecific synthesis of fluorine-18-labelled vinyl fluoride using [18F]fluorine reacted with silane. International Journal of Applied Radiation and Isotopes (1985), 36(12), 953-6.

In certain embodiments, the ethylene reagent is ethylene-1,2-¹³C₂ and the labeled terminal olefin has the structure:

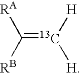

In certain embodiments, the ethylene reagent is ethylene-1,2-¹⁴C₂ and the labeled terminal olefin has the structure:

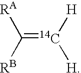

In general, any isotopically labeled ethylene compound, commercially available or synthetically accessible, may be used to practice the invention. For example, labeled ethylene reagents that may be used in practicing the invention include any combination of deuterium-, tritium-, ¹³C-, ¹⁴C-, ¹⁸F-labeled ethylene that can be synthesized. The Angewandte Chemie Int. Ed. Engl. Reference cited above also reports chloro and bromo derivatives. Iodo-labeled ethylene may also be used.

In certain embodiments, 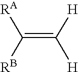
represents a pharmaceutically/therapeutically useful compound. Such compound may be an FDA approved drug, a prodrug, a clinical trial candidate, a lead compound, or a compound at early stages of Research & Development drug discovery program. In certain exemplary embodiments, 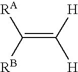
represents one of the compounds depicted in Appendix A, either in free form, known salt form thereof, or any stable salt form the particular compound may be made to exist. In certain exemplary embodiments, 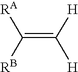
is alminoprofen, amisometradine, dicryl, ethacrynic acid, ethalfluralin, methallatal, rhodinol, acetamidoeugenol, albutoin, alclofenac, alibendol, allethrin I, allethrin II, allocupreide sodium, allylestrenol, almitrine, aloxidone, alpiropride, alprenolol, altrnogest, aminometradine, apiole, aprobarbital, apronalide, bialamicol, butalbital, buthalital sodium, cabergoline, enallylpropymal, enilconazole, eugenol, gravitol, honokiol, isophytol, levallorphan, nalorphine, naloxone, nealbarbital, penicillin O., phenallymal, proxibarbal, rocuronium, safrole, secobarbital sodium, tacrolimus, talbutal, talipexole, thiamylal, valdetamide, veralipride, vigabatrin, verteporfin, bexarotene, calcipotriol, cefdinir, cefixime, exemestane, nalmefene, doxercalciferol, or a compound having the structure:

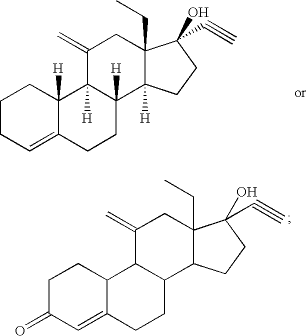

or salt thereof.

In certain embodiments, 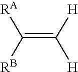
represents a halichondrin-type compound having the structure:

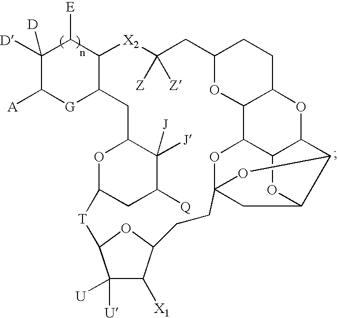

wherein A is a linear or branched C₁₋₆ saturated or branched C₂₋₆ unsaturated hydrocarbon moiety, optionally substituted with between 1 and 13 substituents, preferably between 1 and 10 substituents, wherein at least one substituent is selected from cyano, halo, azido, oxo and Q₁; wherein each occurrence of Q₁ is independently —WR^{W1} wherein W is —O—, —S—, —NR^{W2}—, —CO—, —SO—, —SO₂—, —OSO₂—, —C(═O)O—, —C(═O)NR^{W2}—, —OC(═O)—, —NR^{W2}C(═O)—, —NR^{W2}C(═O)C(═O)—, —NR^{W2} C(═O)NR^{W2}, —NR^{W2}C(═O)O, —OC(═O)NR^{W2}, or —SO₂NR^{W2}—, and R^{W1} and R^{W2} are independently hydrogen, an aliphatic, alicyclic, heteroaliphatic, heterocyclic, aromatic or heteroaromatic moiety;

D and D′ are independently R^{D1} or OR^{D1}, wherein R^{D1} is H, C₁₋₃alkyl, or C₁₋₃haloalkyl;

n is 0 or 1;

E is H, an aliphatic, alicyclic, heteroaliphatic, heterocyclic, aromatic or heteroaromatic moiety, or —WR^{W1} wherein W is —O—, —S—, —NR^{W2}—, —CO—, —SO—, —SO₂—, —OSO₂—, —C(═O)O—, —C(═O)NR^{W2}—, —OC(═O)—, —NR^{W2}C(═O)—, —NR^{W2}C(═O)C(═O)—, —NR^{W2}C(═O)NR^{W2}, —NR^{W2}C(═O)O, —OC(═O)NR^{W2}, or —SO₂NR^{W2}—;

G is O, S, CH₂ or NR^{G};

J and J′ are independently H, C₁₋₆alkoxy, or C₁₋₆alkyl; or J and J′ taken together are ═CH₂ or —O-(straight or branched C₁₋₅alkylene or alkylidene)-O—;

Q is lower alkyl;

T is ethylene, optionally substituted with (CO)OR^{T}, where R^{T} is H or C₁₋₆alkyl;

U and U′ are independently H, C₁₋₆alkoxy, or C₁₋₆alkyl; or U and U′ taken together are ═CH₂ or —O-(straight or branched C₁₋₅alkylene or alkylidene)-O—;

X₁ is H or C₁₋₆alkoxy;

X₂ is O, S, NR^{X2} or CYY′; wherein Y and Y′ is independently H or C₁₋₆alkoxy; or Y and Y′ taken together are ═O, ═CH₂, or —O-(straight or branched C₁₋₅alkylene or alkylidene)-O-; and R^{X2} is hydrogen, alkyl, heteroalkyl, acyl, aryl or heteroaryl; and

Z and Z′ are independently H or C₁₋₆alkoxy; or Z and Z′taken together are ═O, ═CH₂, or —O-(straight or branched C₁₋₅alkylene or alkylidene)-O—;

wherein at least one of (U, U′) or (J, J′) represents ═CH₂.

In certain embodiments, any olefin moiety present in A where A is a branched C₂₋₆ unsaturated hydrocarbon moiety, is a tri- or tetra-substituted olefin moiety.

In certain embodiments, E is R^{E} or OR^{E}, wherein R^{E} is alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, aryl, heteroaryl, -(alkyl)aryl, -(alkenyl)aryl, -(alkynyl)aryl, -(heteroalkyl)aryl, -(heteroalkenyl)aryl, -(heteroalkynyl)aryl, -(alkyl)heteroaryl, -(alkenyl)heteroaryl, -(alkynyl)heteroaryl, -(heteroalkyl)heteroaryl, -(heteroalkenyl)heteroaryl, -(heteroalkynyl)heteroaryl. In certain embodiments, any olefin moiety present in the alkenyl, cycloalkenyl, heteroalkenyl, heterocycloalkenyl, -(alkenyl)aryl, -(alkenyl)aryl, -(heteroalkenyl)aryl, -(alkenyl)heteroaryl, -(heteroalkenyl)heteroaryl referred to directly above is a tri- or tetra-substituted olefin moiety.

In certain embodiments, E is R^{E} or OR^{E}, wherein R^{E} is alkyl, alkynyl, cycloalkyl, cycloalkynyl, heteroalkyl, heteroalkynyl, heterocycloalkyl, heterocycloalkynyl, aryl, heteroaryl, -(alkyl)aryl, -(alkynyl)aryl, -(heteroalkyl)aryl, -(heteroalkynyl)aryl, -(alkyl)heteroaryl, -(alkynyl)heteroaryl, -(heteroalkyl)heteroaryl, -(heteroalkynyl)heteroaryl.

In certain embodiments, G is O.

In certain embodiments, the number of substituents on A can be, for example, between 1 and 6, 1 and 8, 2 and 5, or 1 and 4. Throughout the disclosure, numerical ranges are understood to be inclusive.

In certain embodiments, as applied to Q₁, R^{W1} and R^{W2} are independently H, C₁₋₆alkyl, C₁₋₆haloalkyl, C₁₋₆hydroxyalkyl, C₁₋₆aminoalkyl, C₆₋₁₀aryl, C₆₋₁₀haloaryl (e.g., p-fluorophenyl or p-chlorophenyl), C₆₋₁₀hydroxyaryl, C₁₋₄alkoxy-C₆₋₁₀aryl (e.g., p-methoxyphenyl, 3,4,5-trimethoxyphenyl, p-ethoxyphenyl, or 3,5-diethoxyphenyl), C₆₋₁₀aryl-C₁₋₆alkyl (e.g., benzyl or phenethyl), C₁₋₆alkyl-C₆₋₁₀aryl, C₆₋₁₀haloaryl-C₁₋₆alkyl, C₁₋₆alkyl-C₆₋₁₀haloaryl, (C₁₋₃alkoxy-C₆₋₁₀aryl)-C₁₋₃alkyl, C₂₋₉heterocyclic radical, C₂₋₉heterocyclic radical-C₁₋₆alkyl, C₂₋₉heteroaryl, and C₂₋₉heteroaryl-C₁₋₆alkyl.

In certain embodiments, one of D and D′ is H. In certain embodiments, D and D′ are independently methoxy, methyl, ethoxy, and ethyl. In certain exemplary embodiments, Q is methyl.

In certain embodiments, A is 2,3-dihydroxypropyl, 2-hydroxyethyl, 3-hydroxy-4-perfluorobutyl, 2,4,5-trihydroxypentyl, 3-amino-2-hydroxypropyl, 1,2-dihydroxyethyl, 2,3-dihyroxy-4-perflurobutyl, 3-cyano-2-hydroxypropyl, 2-amino-1-hydroxyethyl, 3-azido-2-hydroxypropyl, 3,3-difluoro-2,4-dihydroxybutyl, 2,4-dihydroxybutyl, 2-hydroxy-2(p-fluorophenyl)-ethyl, —CH₂(CO)(substituted or unsubstituted aryl), —CH₂(CO)(alkyl or substituted alkyl, such as haloalkyl or hydroxyalkyl), or protected form thereof.

In certain embodiments, Q₁ is —NH(CO)(CO)-(heterocyclic radical or heteroaryl), —OSO₂-(aryl or substituted aryl), —O(CO)NH-(aryl or substituted aryl), aminoalkyl, hydroxyalkyl, —NH(CO)(CO)-(aryl or substituted aryl), —NH(CO)(alkyl)(heteroaryl or heterocyclic radical), O(substituted or unsubstituted alkyl)(substituted or unsubstituted aryl), or —NH(CO)(alkyl)(aryl or substituted aryl).

In certain embodiments, the halichondrin-type compound has the following stereochemistry:

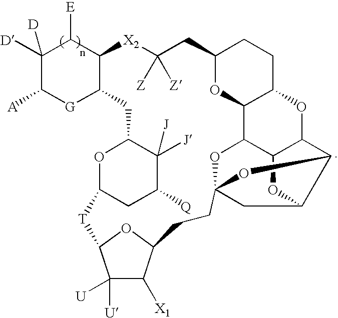

In certain embodiments, the halichondrin-type compound has the structure:

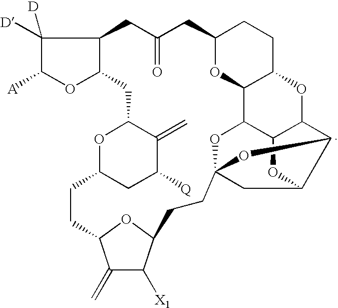

The skilled practitioner will recognize that protection of certain functional groups may be desired prior to labeing a terminal olefin suubstrate (e.g., pharmaceutically/therapeutically useful compound) according to the inventive method. For example, a particular functional moiety, e.g., O, S, or N, may be temporarily “blocked” so that the metathesis reaction can be carried out selectively, and with minimal interference, at another reactive site (e.g., terminal olefin) in a multifunctional compound. Guidance for protecting group chemistry may be found, for example, in “Protective Groups in Organic Synthesis” Third Ed. Greene, T. W. and Wuts, P. G., Eds., John Wiley & Sons, New York: 1999. One of ordinary skill in the art will know how to select suitable protecting groups and reaction conditions to effect protection of functional groups in the terminal olefin substrate, when it is desired.

In general, multiple points of unsaturation (non-aromatic double bonds) in the substrate to be labeled would be expected to undergo the olefin metathesis reaction. Aromatic systems are not affected by metathesis reaction conditions. Therefore, the inventive method may not, in general, be used for labeling terminal olefins in poly-unsaturated substrates without alteration of the substrate at additional non-aromatic unsaturation sites present in the substrate (whether they be terminal or non-terminal olefins). However, this trend is not universal, rather it is substrate dependent. For example, factors such as olefin substitution, type of neighboring functionalities and steric hinderance at additional non-aromatic unsaturation sites present in the substrate, may play a role as to whether these additional non-aromatic unsaturation sites participate in the metathesis reaction. For example, a tri- or tetra-substituted olefin would generally be inert to the reaction conditions. A disubstituted olefin would probably be cleaved, but would be substrate dependent, as is demonstrated herein with respect to certain halicondrin-type compounds. In fact, there is provided herein examples whereby the inventive method allows regiospecific labeling of substrates comprising more than one terminal olefins (e.g., two terminal olefins). For example, deuteriation of each of the three compounds depicted below using the method of the invention proceeded regiospecifically at C-19′. No labeling (deuterium) was detected at C-26′. [Note that no labeling occurred at the fluorene substituent in ER-810951 either].

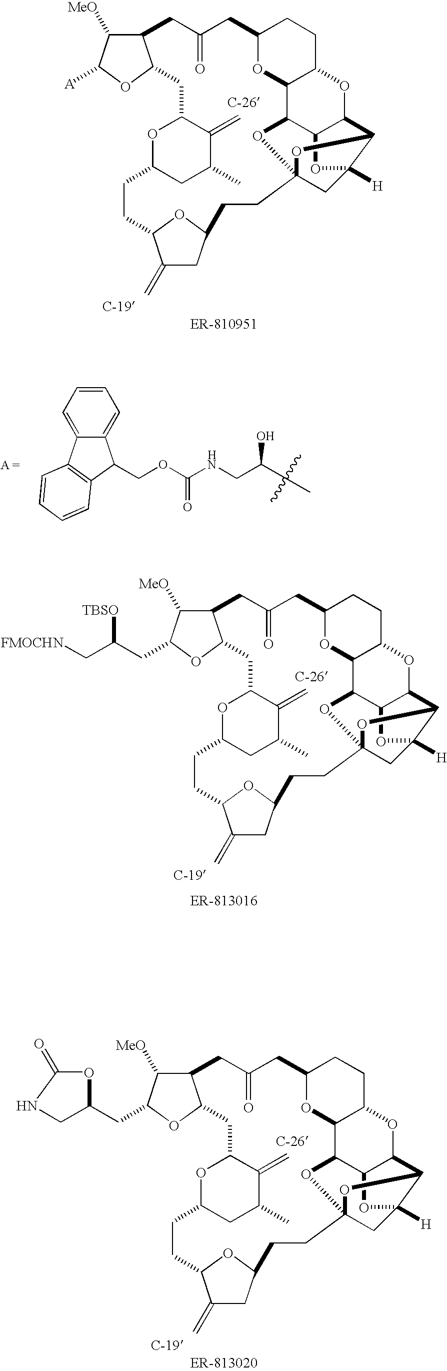

Without wishing to be bound to any particular theory, it is proposed that the macrocyclic ring sterically hinders the C-26′ terminal olefin, and hence renders it inaccessible/unavailable for metathesis reaction. It is likely that regiospecific C-19′ labeling using the method of the present invention can be extended to substrates having the general structure:

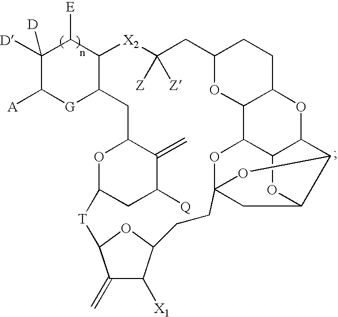

wherein A, D, D′, E, n, G, T, Q, Z, Z′, X¹ and X₂ are as defined herein. In certain embodiments, the inventive method allows regiospecific C-19′ labeling of compounds having the structure: 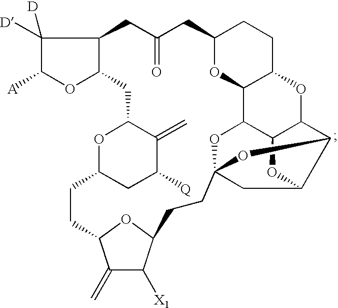
wherein A, D, D′ and Q are as defined herein.

The present invention finds use in any area where isotopically labeled compounds are desired and/or useful. For example, as discussed above, deuterated compounds have been used in pharmaceutical research to investigate the in vivo metabolic fate of the compounds by evaluation of the mechanism of action and metabolic pathway of the non deuterated parent compound. In addition, incorporation of a heavy atom particularly substitution of deuterium for hydrogen, can give rise to an isotope effect that can alter the pharmacokinetics of the drug. This effect is usually insignificant if the label is placed in a molecule at the metabolically inert position of the molecule. For example, deuteration of rapamycin has been reported to result in altered physicochemical and pharmacokinetic properties which enhance its usefulness in the treatment of transplantation rejection, host vs. graft disease, graft vs. host disease, leukemia/lymphoma, hyperproliferative vascular disorders, autoimmune diseases, diseases of inflammation, solid tumors, and fungal infections (See, for example, U.S. Pat. No.: 6,710,053). Thus, the present invention may find use in pharmaceutical research.

Specifically, stable isotope labeling of a drug can alter its physico-chemical properties such as pKa and lipid solubility. These changes may influence the fate of the drug at different steps along its passage through the body. Absorption, distribution, metabolism or excretion can be changed. Absorption and distribution are processes that depend primarily on the molecular size and the lipophilicity of the substance.

Drug metabolism can give rise to large isotopic effect if the breaking of a chemical bond to a deuterium atom is the rate limiting step in the process. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. In any reaction in which the breaking of this bond is the rate limiting step, the reaction will proceed slower for the molecule with the heavy isotope due to kinetic isotope effect. A reaction involving breaking a C-D bond can be up to 700 per cent slower than a similar reaction involving breaking a C—H bond.

More caution should be exercised when using deuterium labeled drugs. If the C-D bond is not involved in any of the steps leading to the metabolite, there may not be any effect to alter the behavior of the drug. If a deuterium is placed at a site involved in the metabolism of a drug, an isotope effect will be observed only if breaking of the C-D bond is the rate limiting step. There is evidence to suggest that whenever cleavage of an aliphatic C—H bond occurs, usually by oxidation catalyzed by a mixed-function oxidase, replacement of the hydrogen by deuterium will lead to observable isotope effect. It is also important to understand that the incorporation of deuterium at the site of metabolism slows its rate to the point where another metabolite produced by attack at a carbon atom not substituted by deuterium becomes the major pathway by a process called “metabolic switching”.

It is also observed that one of the most important metabolic pathways of compounds containing aromatic systems is hydroxylation leading to a phenolic group in the 3 or 4 position to carbon substituents. Although this pathway involves cleavage of the C—H bond, it is often not accompanied by an isotope effect, because the cleavage of this bond is mostly not involved in the rate-limiting step. The substitution of hydrogen by deuterium at the stereo center will induce a greater effect on the activity of the drug.

Clinically relevant questions include the toxicity of the drug and its metabolite derivatives, the changes in distribution or elimination (enzyme induction), lipophilicity which will have an effect on absorption of the drug. Replacement of hydrogen by deuterium at the site involving the metabolic reaction will lead to increased toxicity of the drug. Replacement of hydrogen by deuterium at the aliphatic carbons will have an isotopic effect to a larger extent. Deuterium placed at an aromatic carbon atom, which will be the site of hydroxylation, may lead to an observable isotope effect, although this is less often the case than with aliphatic carbons. But in few cases such as in penicillin, the substitution on the aromatic ring will induce the restriction of rotation of the ring around the C—C bond leading to a favorable stereo-specific situation to enhance the activity of the drug.

Approaching half a century of stable-isotope usage in human metabolic studies has been without documented significant adverse effect. Side-effects with acute D dosing are transitory with no demonstrated evidence of permanent deleterious action. The threshold of D toxicity has been defined in animals and is far in excess of concentrations conceivably used in human studies (Jones P J, Leatherdale S T Clin Sci (Colch) 1991 Apr; 80 (4):277-280). The possibility that D may have additional beneficial pharmacological applications cannot be excluded. For isotopes other than D, evidence of observed toxicity remains to be produced even at dosages far in excess of the range used in metabolic studies. Absence of adverse effect may be attributable to small mass differences and the similar properties of tracer and predominantly abundant isotopes. The precision of extrapolating toxicity thresholds from animal studies remains unknown. However, should perturbation of the delicate homoeostatic characteristic of living organisms occur with use of stable isotopes, it is almost undoubtedly at some level of administration greatly in excess of those administered currently in biomedical research.

Biomedical applications of 14C

¹⁴C is used as a radioactive tracer in clinical nuclear medicine and it is used in different contexts in medical research and when testing new pharmaceuticals on volunteers. In clinical medicine, organic compounds labelled with ¹⁴C are used to demonstrate metabolic abnormalities. One way of carrying out these studies is to use breath tests [G. W. Hepner, Gastroenterology 67 (1974) 1250]. The ¹⁴C-labelled compound is ingested and metabolized, resulting in the end-product carbon dioxide, which is exhaled and easily collected for measurement. The decay of the radionuclide is usually measured by gas flow counters or liquid scintillators and the activity of the sample reveals the degree of, for example, fat malabsorption. Clinically useful information is obtained from samples taken a few hours after the administration of the test compound, even if the total turnover time is much longer. A complete biokinetic study, needed for such purposes as the calculation of the radiation dose, requires sampling for a much longer time, up to several months or even longer.

There are significant uncertainties in the current estimates of the absorbed doses to the body from ¹⁴C-labelled pharmaceuticals, mainly due to the long half-life of ¹⁴C and the difficulties involved to successfully measure with high sensitivity the long-term retention of ¹⁴C in the body. Standard measuring methods, used in medical applications, are only capable of detecting increased levels of ¹⁴C in expired air for a few days after ingestion. There is thus a need for a much more sensitive technique, such as AMS, for a complete study. Accelerator mass spectrometry (AMS) is a relatively new detection technique (first introduced in 1977) which constitutes a highly sensitive method for counting atoms and it is used for detecting very low concentrations of mainly long-lived radionuclides (or stable isotopes) in small samples. The fact that AMS counts atoms rather than decays results in great advantages compared to radiometrical techniques, such as highly reduced sample sizes and shortened measuring times. The AMS technique has been used to study the long-term retention of ¹⁴C after a fat-malabsorption test (using ¹⁴C-labelled triolein) by analysis of expired air [K. Stenström, S. Leide-Svegborn, B. Erlandsson, R. Hellborg, S. Mattsson, L.-E. Nilsson, B. Nosslin, G. Skog and A. Wiebert, Journal of Applied Radiation and Isotopes 47:4 (1996) 417]. Studies are also being performed on the long-term retention of ¹⁴C after a ¹⁴C-urea test [K. Stenström, S. Leide-Svegborn, B. Erlandsson, R. Hellborg, S. Mattsson, L.-E. Nilsson, B. Nosslin and G. Skog, Nucl. Instr. and Meth. B123 (1997) 245-248], which is used to demonstrate abnormal activity of gastrointestinal bacteria. For the urea study, expired air is analysed and a sample preparation procedure for urine samples is under development.

Microdosing

Human microdosing (Human Phase 0) relies on the ultrasensitivity of AMS, and allows to conduct a full human metabolism study (PK, AUC, t_{1/2}, Cₘₐₓ, tₘₐₓ, V_{d}) after administration of as little as 0.5 microgram of drug substance. More typically, however, 100 micrograms of drug are administered. In microdosing one or more drug candidates are taken into humans at trace doses in order to obtain early ADME and PK information. This information is then used as part of the decision tree to select which of the microdosed drugs has the appropriate PK parameters to take further. The aim of these low dose screening ADME studies is to ensure that drugs do not have to be dropped later down the development pathway because of inappropriate metabolism, e.g., first pass, too short a half-life, poor bioavailability etc. As many as one drug in three will be dropped at the Phase I stage of drug development because of PK, pharmacodynamic or toxicity issues. Human microdosing aims to reduce attrition at Phase I.

Selection of a drug from a number of candidates (sometimes a large number as found from high throughput screening or combinatorial chemistry) for further development is usually made in the absence of in vivo human PK data. Currently to reach this stage of human administration (Phase I) as much as 12-18 months preclinical development work is needed. This involves a host of different activities including scale-up of drug production, preclinical toxicology, GMP manufacture, animal ADME studies etc and the expenditure of several million dollars. If one could obtain early human PK information as with microdosing, then candidate selection for full clinical studies should be much improved. This provides the rationale for CBAMS microdosing approach using AMS.

In the microdosing approach several lead candidates are lightly ¹⁴C-labelled and administered to human volunteers at doses from as little as one microgram to up to 100 micrograms. Blood, urine and faecal samples are collected over time and the resulting samples analysed for ¹⁴C content by AMS to determine the t_{1/2}, AUC, V_{d} and Cₘₐₓ. Parent drug concentrations are analysed by HPLC separations of plasma extracts and AMS analysis of the parent drug fraction only.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The method of this invention can be understood further by the examples that illustrate some of the processes by which the inventive method may be practiced. It will be appreciated, however, that these examples do not limit the invention. Variations of the invention, now known or further developed, are considered to fall within the scope of the present invention as described herein and as hereinafter claimed.

General Synthetic Overview

The practitioner has a well-established literature of olefin metathesis chemistry to draw upon, in combination with the information contained in the example which follows, for guidance on synthetic strategies, protecting groups, and other materials and methods useful for specific labeling of terminal olefins with stable, as well as radioactive isotopes via olefin metathesis. The method may be practiced according to the synthetic method described herein using any of the available relevant chemical transformations, combined with protection and deptrotection as desired or required. The various starting materials are either commercially available or may be obtained by standard procedures of organic chemistry. The preparation of certain starting materials (e.g., halichondrin core) is described elsewhere (See, for example, U.S. Pat. No. 6, 214,865).

General Reaction Procedures

Unless mentioned specifically, reaction mixtures were stirred using a magnetically driven stirrer bar. An inert atmosphere refers to either dry argon or dry nitrogen. Reactions were monitored either by thin layer chromatography, by proton nuclear magnetic resonance or by high-pressure liquid chromatography (HPLC), of a suitably worked up sample of the reaction mixture. Analysis of incorporation is determined using mass spectrometry and liquid scintillation counting.

### EXAMPLE 1

### C-19′ Deuteration of Halichondrin Analog ER-810951

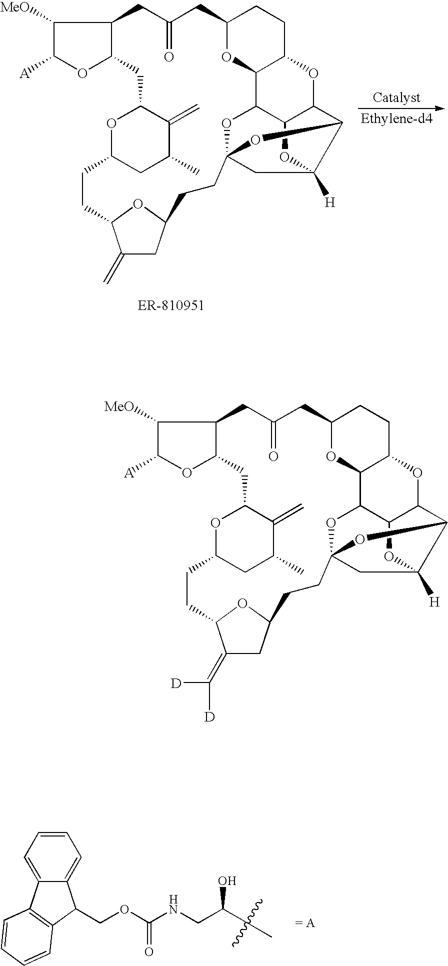

ER-810951 (1 wt, 1 eq) and Grubb's 2^{nd} generation olefin metathesis catalyst (1.67 wt, 1.5 eq) were placed in a 25 mL Schlenk-type vessel. The atmosphere was exchanged for nitrogen gas three times, then evacuated. Ethylene-d4 (25 mL, 800 eq.) was charged into the evacuated vessel. Dichloromethane 92 mL was charged into the vessel. The vessel was sealed and placed in a 35° C. bath. The mixture was stirred for 18 hours. The reaction mixture was cooled to room temperature, and the vessel was opened. The mixture was purified by flash chromatography. The product was concentrated to give a white-brown solid (0.3 wt, 0.3 eq.). Analysis of the product by ¹H NMR (CD₃OD), d1=3 sec showed incorporation at the C₁₉ methylene approaching 75%. MS analysis (M+Na, 954) showed significant enrichment of M+2, also approaching 75-80%.

### EXAMPLE 2

### C-19′ Carbon-14 Labeling of Halichondrin Analog ER-813018

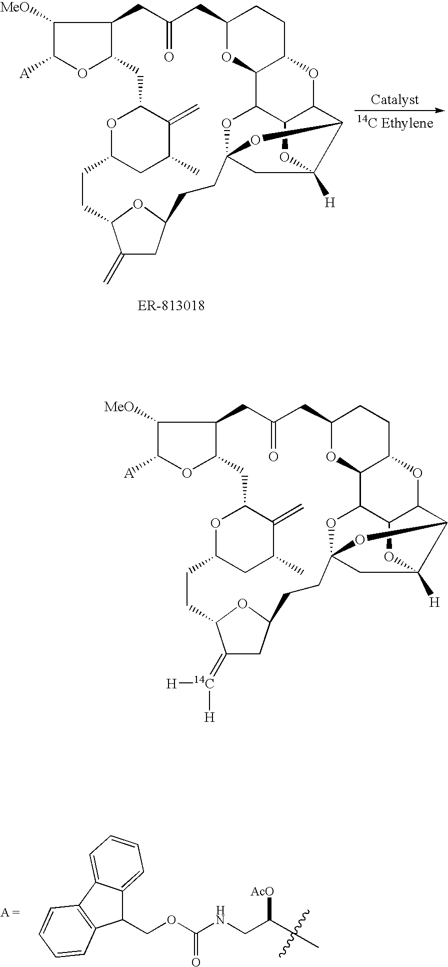

A reaction flask was charged with ER-813018 (1 wt, 1 equiv.).* Toluene (100 vol) was added. The solution was frozen with liquid nitrogen, vacuum was applied, and the solution was thawed. The reaction mixture was re-frozen with liquid nitrogen, and 1,2-¹⁴C-ethylene (2-3 equiv.) was transferred to the reaction vessel. The vessel was sealed and warmed to room temperature. The reaction mixture was heated to 60-65° C. After the desired temperature was reached, a solution of Grubbs ₂nd generation catalyst (5 mol %) in toluene was added to the reaction mixture. The resulting mixture was stirred for 20-60 minutes. The reaction was cooled.** The reaction mixture was sampled and analyzed by mass spectrometry versus an unlabeled standard to determine the specific activity. The above process was repeated until the desired level of incorporation was achieved.***, ****

* The reaction flask size was determined such that the reaction occurred at <1 atm internal pressure. The volume of gas and partial pressure of solvent are taken into account in the determination.

** Additional steps were required, which included removal of “hot” ethylene.

*** All additional charges were added based upon amount of starting material, not conversion remaining.

* In between charges of ethylene, it it was advantageous to conduct a flash chromatographic purification to remove catalyst decomposition products. This improved the yield of the reaction. For example, in initial trial runs, a series of 3 charges of 2.9 equiv. ethylene and 5 mol % catalyst solution was followed by purification. The purified material was resubjected to 3 more charges of 2.9 equiv. ethylene and 5 mol % catalyst, and then purified a final time. In the first cycle of charges: 1. 3×5 mol % catalyst charge

2. 3×2.9 equiv. ethylene (specific activity of 116 mCi/mmol)

3. specific activity determined by MS=30 mCi/mmol (˜50 mol % incorporation)

4. flash chromatography purification

5. final isolated material: 0.83 wt material yield, 2.1 mCi total activity, specific activity of 30 mCi/mmol corresponding to 0.07 mmol of 70% yield. 96% HPLC area purity.

In the 2^{nd} cycle:

1. 3×5 mol % catalyst charge

2. 3×2.9 equiv. ethylene

3. IPC (after charge 1), specific activity=39 mCi/mmol (˜67 mol % incorporation)

4. IPC (after charge 2), specific activity=45 mCi/mmol (˜78 mol % incorporation)

5. IPC (after charge 3), specific activity=50 mCi/mmol (˜86 mol % incorporation)

6. flash chromatography purification

7. final isolated material: 0.73 wt material yield, 3.1 mCi total activity, specific activity of 50 mCi/mmol corresponding to 0.062 mmol of 62% yield. 92.5% HPLC area purity.

A larger scale run afforded a better yield and similar specific activity, however it incorporated a different charge and purification cycle. A series of 2 charges of 2.5 equiv 1,2-14C-ethylene and 2 charges of 5 mol % catalyst (toluene solution) was followed by purification. This procedure was repeated two more times. This yielded product of 0.87 wt, 17.1 mCi total activity, at a specfic activity of 46 mCi/mmol.

### EXAMPLE 3

### C-19′ Carbon-14 Labeling of Halichondrin Analogs ER-813016 and ER-813020

C-19′ ¹³C labeling was carried out using procedure similar to that described in Example 2 with the following substrates:

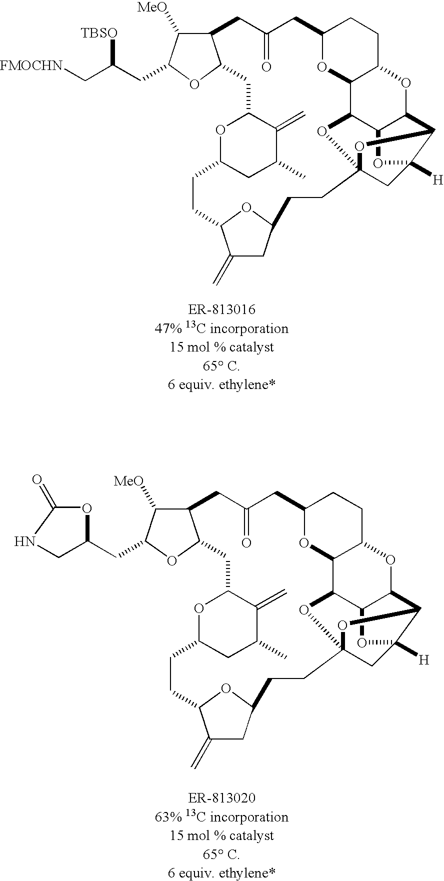

### EXAMPLE 4

### C-19′ Carbon-13 Labeling of Halichondrin Intermediate ER-804028

¹³C labeling using a similar procedure was carried out with the following substrate:

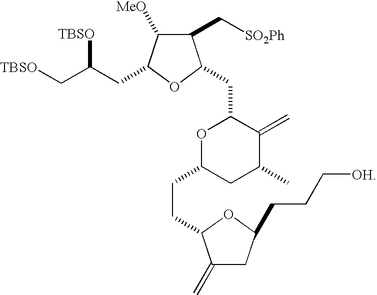

¹³C incorporation was comparable to that observed for other substrates:

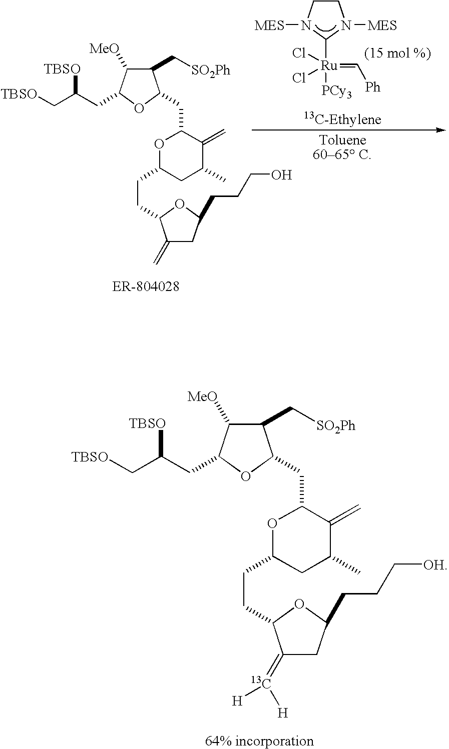

What is claimed is:

1. A method for isotopically labeling a terminal olefin, the method comprising a step of treating a terminal olefin substrate having the structure: 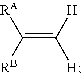
with a labeled ethylene reagent in the presence of a suitable catalyst under suitable olefin metathesis reaction conditions to form a labeled terminal olefin having the structure: 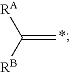
wherein R^{A} and R^{B} are independently hydrogen, or an aliphatic, alicyclic, heteroaliphatic, heterocyclic, aromatic or heteroaromatic moiety, with the proviso that R^{A} and R^{B} are not each hydrogen, or R^{A} and R^{B} taken together with the carbon atom to which they are attached form an alicyclic or heterocyclic moiety; and
* denotes the presence of an isotopic label on the terminal carbon atom.

2. The method of claim 1 wherein R^{A} and R^{B} are independently hydrogen, or an aliphatic, alicyclic, heteroaliphatic, heterocyclic, aryl or heteroaryl moiety, with the proviso that R^{A} and R^{B} are not each hydrogen, or R^{A} and R^{B} taken together with the carbon atom to which they are attached form an alicyclic or heterocyclic moiety.

3. The method of claim 1 wherein more than one metathesis cycle may be desired to obtain the target conversion rate.

4. The method of claim 3 comprising steps of:
(a) treating a terminal olefin substrate having the structure: 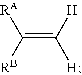
with a labeled ethylene reagent in the presence of a suitable catalyst under suitable olefin metathesis reaction conditions to yield a reaction mixture comprising a labeled terminal olefin having the structure: 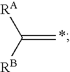
and unreacted terminal olefin substrate;
wherein R^{A} and R^{B} are independently hydrogen, or an aliphatic, alicyclic, heteroaliphatic, heterocyclic, aromatic or heteroaromatic moiety, with the proviso that R^{A} and R^{B} are not each hydrogen, or R^{A} and R^{B} taken together with the carbon atom to which they are attached form an alicyclic or heterocyclic moiety;
* denotes the presence of an isotopic label on the terminal carbon atom; and
(b) repeating step (a) using the reaction mixture as a substrate, thereby reducing the amount of unreacted terminal olefin substrate;
(c) optionally repeating step (b) until the ratio [labeled terminal olefin]/[unreacted terminal olefin substrate] reaches a desired value.

5. The method of claim 1 wherein neither R^{A} nor R^{B} comprises an olefin moiety.

6. The method of claim 1 wherein neither R^{A} nor R^{B} comprises a disubstituted olefin moiety.

7. The method of claim 1 wherein R^{A} and R^{B} are independently alkyl, alkynyl, cycloalkyl, cycloalkynyl, heteroalkyl, heteroalkynyl, heterocycloalkyl, heterocycloalkynyl, aryl, heteroaryl, -(alkyl)aryl, -(alkynyl)aryl, -(heteroalkyl)aryl, -(heteroalkynyl)aryl, -(alkyl)heteroaryl, -(alkynyl)heteroaryl, -(heteroalkyl)heteroaryl, -(heteroalkynyl)heteroaryl, or R^{A} and R^{B} taken together with the carbon atom to which they are attached form an alicyclic or heterocyclic moiet.

8. The method of claim 1 wherein the catalyst is Grubb's 2^{nd} generation olefin metathesis catalyst.

9. The method of claim 1 wherein the ethylene reagent is ethylene-d4 and the labeled terminal olefin has the structure:

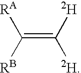

10. The method of claim 1 wherein the ethylene reagent is ethylene-³H₄ and the labeled terminal olefin has the structure:

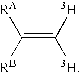

11. The method of claim 1 wherein the ethylene reagent is ethylene-1,2-¹³C₂ and the labeled terminal olefin has the structure:

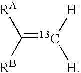

12. The method of claim 1 wherein the ethylene reagent is ethylene-1,2-¹⁴C₂ and the labeled terminal olefin has the structure:

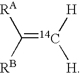

13. The method of claim 1 wherein 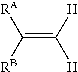
represents a pharmaceutically/therapeutically useful compound.

14. The method of claim 13 wherein 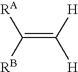
is alminoprofen, amisometradine, dicryl, ethacrynic acid, ethalfluralin, methallatal, rhodinol, acetamidoeugenol, albutoin, alclofenac, alibendol, allethrin I, allethrin II, allocupreide sodium, allylestrenol, almitrine, aloxidone, alpiropride, alprenolol, altrnogest, aminometradine, apiole, aprobarbital, apronalide, bialamicol, butalbital, buthalital sodium, cabergoline, enallylpropymal, enilconazole, eugenol, gravitol, honokiol, isophytol, levallorphan, nalorphine, naloxone, nealbarbital, penicillin O., phenallymal, proxibarbal, rocuronium, safrole, secobarbital sodium, tacrolimus, talbutal, talipexole, thiamylal, valdetamide, veralipride, vigabatrin, verteporfin, bexarotene, calcipotriol, cefdinir, cefixime, exemestane, nalmefene, doxercalciferol, or a compound having the structure: 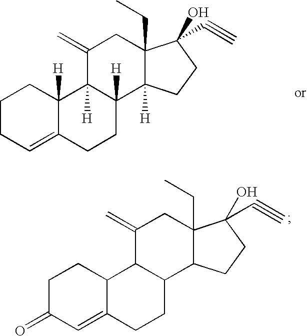
or salt thereof.

15. The method of claim 13 wherein 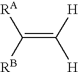
represents a halichondrin-type compound having the structure: 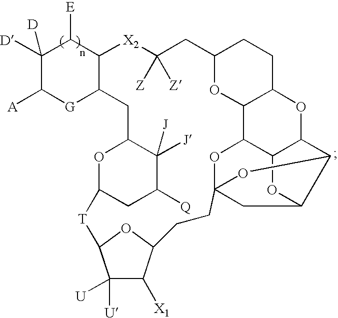
wherein A is a linear or branched C₁₋₆ saturated or branched C₂₋₆ unsaturated hydrocarbon moiety, optionally substituted with between 1 and 13 substituents, preferably between 1 and 10 substituents, wherein at least one substituent is selected from cyano, halo, azido, oxo and Q₁; wherein each occurrence of Q₁ is independently —WR^{W1} wherein W is —O—, —S—, —NR^{W2}—, —CO—, —SO—, —SO₂—, —OSO₂—, —C(═O)O—, —C(═O)NR^{W2}—, —OC(═O)—, NR^{W2}C(⊚O)—, —NR^{W2}C(═O)C(═O), —NR^{W2}C(═O)NR^{W2}, NR^{W2}C(═O)O, —OC(⊚O)NR^{W2}, or —SO₂NR^{W2}—, and R^{W1} and R^{W2} are independently hydrogen, an aliphatic, alicyclic, heteroaliphatic, heterocyclic, aromatic or heteroaromatic moiety;
D and D′ are independently R^{D1} or OR^{D1}, wherein R^{D1} is H, C₁₋₃alkyl, or C₁₋₃haloalkyl;
n is 0 or 1;
E is H, an aliphatic, alicyclic, heteroaliphatic, heterocyclic, aromatic or heteroaromatic moiety, or —WR^{W1} wherein W is —O—, —S—, —NR^{W2}, —CO—, —SO—, —SO₂—, —OSO₂—, —C(═O)O—, —C(═O)NR^{W2}—, —OC(═O)—, —NR^{W2}C(═O)—, —NR^{W2}C(═O)C(⊚O)—, —NR^{W2}C(═O)NR^{W2}, —NR^{W2}C(═O)O, —OC(═O)NR^{W2}, or —SO₂NR^{W2}—;
G is O, S, CH₂ or NR^{G};
J and J′ are independently H, C₁₋₆alkoxy, or C₁₋₆alkyl; or J and J′ taken together are ═CH₂ or —O-(straight or branched C₁₋₅alkylene or alkylidene)-O—;
Q is lower alkyl;
T is ethylene, optionally substituted with (CO)OR^{T}, where R^{T} is H or C₁₋₆alkyl;
U and U′ are independently H, C₁₋₆alkoxy, or C₁₋₆alkyl; or U and U′ taken together are 50 CH₂ or —O-(straight or branched C₁₋₅alkylene or alkylidene)-O—;
X₁ is H or C₁₋₆alkoxy;
X₂ is O, S, NR^{X2} or CYY′; wherein Y and Y′ is independently H or C₁₋₆alkoxy; or Y and Y′ taken together are ═O, ═CH₂, or —O-(straight or branched C₁₋₅alkylene or alkylidene)-O—; and R^{X2} is hydrogen, alkyl, heteroalkyl, acyl, aryl or heteroaryl; and
Z and Z′ are independently H or C₁₋₆alkoxy; or Z and Z′ taken together are ═O, ═CH₂, or —O-(straight or branched C₁₋₅alkylene or alkylidene)-O—;
wherein at least one of (U, U′) or (J, J′) represents ═CH₂.

16. The method of claim 15 wherein E is R^{E} or OR^{E}, wherein R^{E} is alkyl, alkynyl, cycloalkyl, cycloalkynyl, heteroalkyl, heteroalkynyl, heterocycloalkyl, heterocycloalkynyl, aryl, heteroaryl, -(alkyl)aryl, -(alkynyl)aryl, -(heteroalkyl)aryl, -(heteroalkynyl)aryl, -(alkyl)heteroaryl, -(alkynyl)heteroaryl, -(heteroalkyl)heteroaryl, -(heteroalkynyl)heteroaryl.

17. The method of claim 15 wherein G is O.

18. The method of claim 15 wherein R^{W1} and R^{W2}, as applied to Q₁, are independently H, C₁₋₆alkyl, C₁₋₆haloalkyl, C₁₋₆hydroxyalkyl, C₁₋₆aminoalkyl, C₆₋₁₀aryl, C₆₋₁₀haloaryl (e.g., p-fluorophenyl or p-chlorophenyl), C₆₋₁₀hydroxyaryl, C₁₋₄alkoxy-C₆₋₁₀aryl (e.g., p-methoxyphenyl, 3,4,5-trimethoxyphenyl, p-ethoxyphenyl, or 3,5-diethoxyphenyl), C₆₋₁₀aryl-C₁₋₆alkyl (e.g., benzyl or phenethyl), C₁₋₆alkyl-C₆₋₁₀aryl, C₆₋₁₀haloaryl-C₁₋₆alkyl, C₁₋₆alkyl-C₆₋₁₀haloaryl, (C₁₋₃alkoxy-C₆₋₁₀aryl)-C₁₋₃alkyl, C₂₋₉heterocyclic radical, C₂₋₉heterocyclic radical-C₁₋₆alkyl, C₂₋₉heteroaryl, and C₂₋₉heteroaryl-C₁₋₆alkyl.

19. The method of claim 15 wherein one of D and D′ is H.

20. The method of claim 15 wherein D and D′ are independently hydrogen, methoxy, methyl, ethoxy, and ethyl.

21. The method of claim 15 wherein Q is methyl.

22. The method of claim 15 wherein A is 2,3-dihydroxypropyl, 2-hydroxyethyl, 3-hydroxy-4-perfluorobutyl, 2,4,5-trihydroxypentyl, 3-amino-2-hydroxypropyl, 1,2-dihydroxyethyl, 2,3-dihyroxy-4-perflurobutyl, 3-cyano-2-hydroxypropyl, 2-amino-1-hydroxy ethyl, 3-azido-2-hydroxypropyl, 3,3-difluoro-2,4-dihydroxybutyl, 2,4-dihydroxybutyl, 2-hydroxy-2(p-fluorophenyl)-ethyl, —CH₂(CO)(substituted or unsubstituted aryl), —CH₂(CO)(alkyl or substituted alkyl, such as haloalkyl or hydroxyalkyl), or protected form thereof.

23. The method of claim 15 wherein Q₁ is —NH(CO)(CO)-(heterocyclic radical or heteroaryl), —OSO₂-(aryl or substituted aryl), —O(CO)NH-(aryl or substituted aryl), aminoalkyl, hydroxyalkyl, —NH(CO)(CO)-(aryl or substituted aryl), —NH(CO)(alkyl)(heteroaryl or heterocyclic radical), O(substituted or unsubstituted alkyl)(substituted or unsubstituted aryl), or —NH(CO)(alkyl)(aryl or substituted aryl).

24. The method of claim 15 wherein the halichondrin-type compound has the following stereochemistry:

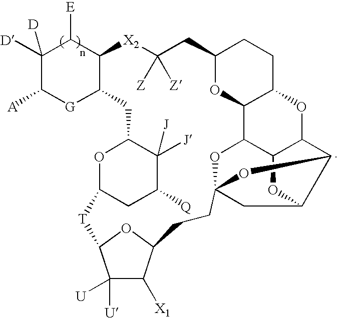

25. The method of claim 15S wherein the halichondrin-type compound has the structure:

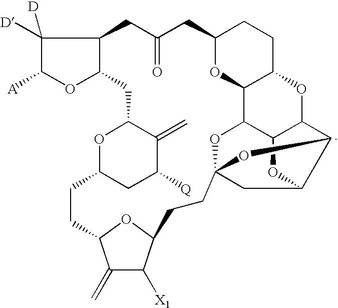

26. The method of claim 25 wherein the halichondrin-type compound has the structure:

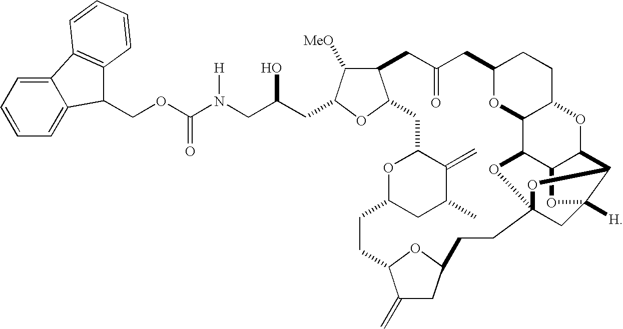

27. The method of claim 25 wherein the halichondrin-type compound has the structure:

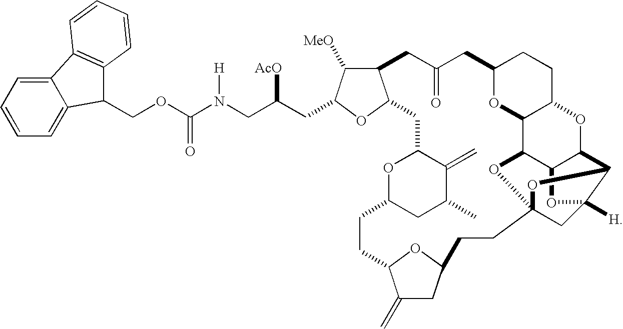

28. The method of claim 25 wherein the halichondrin-type compound has the structure:

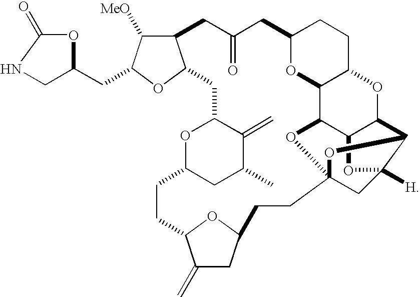

29. The method of claim 25 wherein the halichondrin-type compound has the structure:

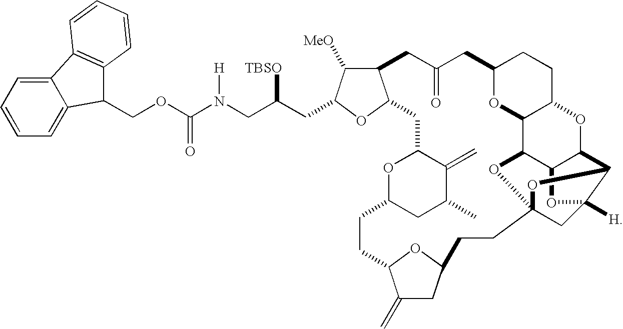

30. The method of claim 1 wherein the terminal olefin substrate has the structure: [chemical structure]
